(19) United States
(12) Patent Application Publication
(10) Pub. No.: US 2013/0316994 A1
(43) Pub. Date: Nov. 28, 2013

(54) Methods of Reducing Risk of Hepatobiliary Dysfunction During Rapid Weight Loss with METAP-2 Inhibitors

(71) Applicant: Thomas E. Hughes, Boston, MA (US)

(72) Inventor: Thomas E. Hughes, Boston, MA (US)

(73) Assignee: Zafgen, Inc., Cambridge, MA (US)

(21) Appl. No.: 13/990,170

(22) PCT Filed: Nov. 29, 2011
(86) PCT No.: PCT/US11/62320

§ 371 Date: Aug. 9, 2013

Related U.S. Application Data
(60) Provisional application No. 61/417,687, filed on Nov. 29, 2010.

(51) Int. Cl. A61K 31/336; A61K 45/06

(52) U.S. Cl. CPC A61K 31/336; A61K 45/06
USPC 514/171; 514/475

(57) ABSTRACT

The invention generally relates in part to methods of reducing hepatobiliary dysfunction and reducing risk of incident hepatobiliary dysfunction, comprising administering a MetAP2 inhibitor to patients in need thereof. The invention also relates in part to methods of effecting weight loss while reducing hepatic injury or risk thereof, comprising administering a MetAP2 inhibitor.

### CROSS REFERENCE TO RELATED APPLICATION

This application claims the benefit of and priority to U.S. Provisional Patent Application No. 61/417,687, filed Nov. 29, 2010, the content of which is hereby incorporated by reference in its entirety.

### BACKGROUND

Over 1.1 billion people worldwide are reported to be overweight. Obesity is estimated to affect over 90 million people in the United States alone. Twenty-five percent of the population in the United States over the age of twenty is considered clinically obese. While being overweight or obese presents problems (for example restriction of mobility, discomfort in tight spaces such as theater or airplane seats, social difficulties, etc.), these conditions, in particular clinical obesity, affect other aspects of health, i.e., diseases and other adverse health conditions associated with, exacerbated by, or precipitated by being overweight or obese. The estimated mortality from obesity-related conditions in the United States is over 300,000 annually (O'Brien et al. (2002) Amer J Surgery 184:4S-8S; and Hill et al. (1998) Science 280:1371).

There is no curative treatment for being overweight or obese. Traditional pharmacotherapies for treating an overweight or obese subject, such as serotonin and noradrenergic re-uptake inhibitor, noradrenergic re-uptake inhibitors, selective serotonin re-uptake inhibitors, intestinal lipase inhibitors, or surgeries such as stomach stapling or gastric banding, have been shown to provide minimal short-term benefits or significant rates of relapse, and have further shown harmful side-effects to patients.

Even when traditional pharmacotherapies and/or surgeries are successful, clinical studies have shown that weight loss, particularly rapid weight loss, is associated with the formation of gallstones. In studies of patients receiving supervised very low calorie diet therapy, 12.1% developed gallstones during or shortly after therapy. In studies of patients having gastric bypass surgery, 37.8% of patients developed gallstones within 12-18 months of surgery (Everhart (1993) Ann Intern Med 119(10):1029-1035).

MetAP2 encodes a protein that functions at least in part by enzymatically removing the amino terminal methionine residue from certain newly translated proteins such as glyceraldehyde-3-phosphate dehydrogenase (Warder et al. (2008) J Proteome Res 7:4807)), and in part by modulating the function of phosphorylated regulatory proteins such as ERK1/2 (Datta et al., (2004) Biochemistry 43:14821). Increased expression of the MetAP2 gene has been historically associated with various forms of cancer. Molecules inhibiting the enzymatic activity of MetAP2 have been identified and have been explored for their utility in the treatment of various tumor types (Wang et al. (2003) Cancer Res 63:7861) and infectious diseases such as microsporidiosis, leishmaniasis, and malaria (Zhang et al. (2002) J Biomed Sci.9:34). Notably, inhibition of MetAP2 activity in obese and obese-diabetic animals leads to a reduction in body weight in part by increasing the oxidation of fat and in part by reducing the consumption of food (Rupnick et al. (2002) Proc Natl Acad Sci USA 99:10730).

Such MetAP2 inhibitors may be useful as well for patients with excess adiposity and conditions related to adiposity including type 2 diabetes, hepatic steatosis, and cardiovascular disease (e.g., by ameliorating insulin resistance, reducing hepatic lipid content, and reducing cardiac workload). Accordingly, compounds capable of modulating MetAP2 are needed to address the treatment of obesity and related diseases as well as other ailments favorably responsive to MetAP2 modulator treatment. Further, a need exists for compounds that effectively treat obesity without the corresponding risk of hepatobiliary dysfunction.

### SUMMARY

At least in part, this disclosure is based on the surprising discovery that administering a pharmaceutically effective amount of a MetAP2 inhibitor to a patent in need thereof results in rapid weight loss without the common side effect of hepatobiliary dysfunction, for example, gallstone formation.

The disclosure is in part directed to a method of inducing rapid weight loss in a patient in need thereof, comprising administering to said patient a pharmaceutically effective amount of a MetAP2 inhibitor, wherein said patient has reduced incidence of hepatobiliary dysfunction as compared to a subject with rapid weight loss after treatment by a reduced energy diet or bariatric surgery. Such biliary tract disorders may be selected from the group consisting of biliary sludge, pancreatitis, or cholelithiasis.

The disclosure is also related to a method of reducing the risk of gallstone formation in a patient being treated for obesity and undergoing rapid weight loss, comprising administering a pharmaceutically effective amount of a MetAP2 inhibitor.

In one aspect, the disclosure relates to a method of treating obesity in a patient in need thereof comprising administering a MetAP2 inhibitor, wherein said method results in rapid weight loss and a reduced risk of gallstone formation in said patient, as compared to the risk of forming gallstones in a subject administered a reduced energy diet or bariatric surgery.

The disclosure is further related to a method of reducing hepatobiliary dysfunction, as indicated by reductions in circulating plasma alkaline phosphatase levels in a patient undergoing rapid weight loss, comprising administering a pharmaceutically effective amount of a MetAP2 inhibitor.

In some embodiments, the rapid weight loss is at least about 20 percent of excess body weight after 19 weeks of treatment with the MetAP2 inhibitor. In other embodiments, the rapid weight loss is at least about 25 percent of excess body weight after 25 weeks of treatment with the MetAP2 inhibitor.

In one aspect, the disclosure relates to a method of treating or reducing the risk of developing liver tumors in a patient in need thereof, comprising administering a pharmaceutically effective amount of a MetAP2 inhibitor to the patient.

In another aspect, the disclosure relates to a method of treating hepatobiliary dysfunction in a patient in need thereof, comprising administering a pharmaceutically effective amount of a MetAP2 inhibitor, wherein alkaline phosphatase levels in the patient are reduced.

In yet another aspect, the disclosure relates to a method of treating gallstones in a patient in need thereof, comprising administering a pharmaceutically effective amount of a MetAP2 inhibitor.

In some embodiments, the patient is being treated for obesity and undergoing rapid weight loss.

The patient may be, for example, a human, a cat or a dog. The patient may be female. In some embodiments, the human patient has a Body Mass Index measurement of at least about 20 kg/m², 25 kg/m², at least about 30 kg/m², or at least about 40 kg/m².

In other embodiments, the pharmaceutically effective amount of the MetAP2 inhibitor does not substantially modulate or suppress angiogenesis.

MetAP2 inhibitors may be a substantially irreversible inhibitor, e.g., fumagillin, fumagillol or fumagillin ketone, or derivatives thereof, siRNA, shRNA, an antibody, or a antisense compound, or may be a substantially reversible inhibitor. For example, an MetAP2 inhibitor may be selected from O-(4-dimethylaminoethoxycinnamoyl)fumagillol and pharmaceutically acceptable salts thereof.

In some embodiments, the method further comprises administering to a patient a pharmaceutically acceptable amount of a non-steroidal anti-inflammatory agent or ursodeoxycholic acid.

### BRIEF DESCRIPTION OF FIGURES

FIG. 1 depicts the results of administration of a MetAP2 inhibitor to obese patients and shows the reduction in alkaline phosphatase that occurs with increasing dosage.

### DETAILED DESCRIPTION

### Overview

The disclosure relates at least in part to methods for inducing rapid weight loss in an obese patient, wherein said patient has reduced incidence of hepatobiliary dysfunction as compared to a subject with rapid weight loss after treatment by a reduced energy diet or bariatric surgery. For example, provided herein are methods of treating obesity comprising administering to a patient an effective amount of a MetAP2 inhibitor, wherein said method results in rapid weight loss and a reduced risk of gallstone formation in said patient, as compared to the risk of forming gallstones in a subject administered a reduced energy diet or bariatric surgery.

Obesity and being overweight refer to an excess of fat in proportion to lean body mass. Excess fat accumulation is associated with increase in size (hypertrophy) as well as number (hyperplasia) of adipose tissue cells. Obesity is variously measured in terms of absolute weight, weight:height ratio, degree of excess body fat, distribution of subcutaneous fat, and societal and esthetic norms. A common measure of body fat is Body Mass Index (BMI). The BMI refers to the ratio of body weight (expressed in kilograms) to the square of height (expressed in meters). Body mass index may be accurately calculated using the formulas: SI units: BMI=weight(kg)/(height²(m²), or US units: BMI=(weight(b)*703)/(height²(in²).

In accordance with the U.S. Centers for Disease Control and Prevention (CDC), an overweight adult has a BMI of 25 kg/m² to 29.9 kg/m², and an obese adult has a BMI of 30 kg/m² or greater. A BMI of 40 kg/m² or greater is indicative of morbid obesity or extreme obesity. For children, the definitions of overweight and obese take into account age and gender effects on body fat.

BMI does not account for the fact that excess adipose can occur selectively in different parts of the body, and development of adipose tissue can be more dangerous to health in some parts of the body rather than in other parts of the body. For example, “central obesity”, typically associated with an “apple-shaped” body, results from excess adiposity especially in the abdominal region, including belly fat and visceral fat, and carries higher risk of co-morbidity than “peripheral obesity”, which is typically associated with a “pear-shaped” body resulting from excess adiposity especially on the hips. Measurement of waist/hip circumference ratio (WHR) can be used as an indicator of central obesity. A minimum WHR indicative of central obesity has been variously set, and a centrally obese adult typically has a WHR of about 0.85 or greater if female and about 0.9 or greater if male.

Excess body weight may be assessed, for example, by comparing the weight of a patient in need of treatment to the weight of the same patient that would achieve a desired, e.g. non-obese, BMI (e.g. a desired BMI of about 25 or less). For example, excess body weight of a 1.6 m patient weighing 89.6 kg (and having a BMI of 35) may be found by calculating the weight required for a BMI of 25 (i.e., about 64 kg); the initial excess body weight of such patient would about 89.6-64=25.6 kg.

Methods of determining whether a subject is overweight or obese that account for the ratio of excess adipose tissue to lean body mass may involve obtaining a body composition of the subject. Body composition can be obtained by measuring the thickness of subcutaneous fat in multiple places on the body, such as the abdominal area, the subscapular region, arms, buttocks and thighs. These measurements are then used to estimate total body fat with a margin of error of approximately four percentage points. Another method is bioelectrical impedance analysis (BIA), which uses the resistance of electrical flow through the body to estimate body fat. Another method is using a large tank of water to measure body buoyancy. Increased body fat will result in greater buoyancy, while greater muscle mass will result in a tendency to sink. Another method is fan-beam dual energy X-ray absorptiometry (DEXA). DEXA allows body composition, particularly total body fat and/or regional fat mass, to be determined non-invasively.

In obese and/or hyperinsulinemic patients, fatty acid synthesis and sterol (i.e., cholesterol) biosynthesis are elevated secondary to increased energy intake and hyperinsulinemia. Ketone body production and utilization are typically suppressed in an obese patient, potentially reducing hepatic satiety signals and increasing food consumption. However, administration of a MetAP2 inhibitor, without being limited by any theory, leads to activation of adipose tissue lipase activity and/or stimulating production and/or activity of the rate-limiting enzyme of beta-hydroxybutyrate production (3-hydroxymethyl glutaryl CoA synthase), leading to elevated ketone body production. Similarly, without being limited by any theory, administration of a MetAP2 inhibitor suppresses fatty acid and/or cholesterol synthesis, leading to reduced liver fat and cholesterol content. Bile composition may be altered in the setting of MetAP2 inhibitor treatment, improving the solubility of bile contents and leading to reduced sludge and/or gallstone formation.

The coordinated and physiologic induction of anti-obesity activities mediated by the methods of the present invention may lead to a healthy reduction in tissue levels of triglyceride, diacylglycerol, and other fat-related mediators and oxidants, and can result in a new steady state situation that favors lean body composition and increased whole body energy metabolism. Without being bound by any theory, it is believed that the mechanistic cascade activated by MetAP2 inhibitors leads to fat tissue being converted to ketone bodies and burned as fuel, unlike existing therapies (including e.g., calorie or energy restricted diets) that target central control of food intake and that may carry adverse side effects (e.g., adverse neurological side effects). Partly as a consequence of improved metabolic function of the liver, glucose uptake by the liver is improved, reducing glucose concentrations in circulation and reducing the demand for insulin secretion and improving pancreatic beta cell function. Further, therapeutically effective doses contemplated herein will not typically induce any anti-angiogenic action.

Methods provided herein may substantially increase or stabilize glucose tolerance in a patient, for example, may increase glucose deposition as glycogen in a patient such as an overweight, obese, and/or diabetic patient or a patient that has reduced glucose tolerance.

### Hepatobiliary Dysfunction

Hepatobiliary dysfunction may lead to any number of biliary tract disorders, including cholecystitis (inflammation of the gall bladder), biliary sludging, pancreatitis, liver tumors and cholelithiasis (gallstones). The gallbladder functions by storing bile secreted by the liver, and releasing it into the intestine when fats are ingested. Gallstones, which form in the gallbladder, typically consist of hardened deposits of cholesterol or bilirubin and other components of digestive fluid. Gallstones that block the flow of bile from the liver to the intestine may cause abdominal and/or shoulder pain, fever, chills, vomiting, jaundice and abdominal fullness. Complications of gallstones include cholecystitis, cholangitis (bile duct infection), pancreatitis, and gallbladder cancer. Gallbladder disease, including gallstones, can be diagnosed by testing levels of alkaline phosphatase, bilirubin, 5′-nucleotidase or mitochondrial antibodies in a patient's blood. Alkaline phosphatase is an enzyme that is produced in the liver and released into the blood during injury. Elevated levels of alkaline phosphatase are indicative of hepatobiliary injury, including liver cancer, inflammation, gallstones or bile duct obstruction. Treatments for gallstones include surgery (e.g., removal of the gallbladder) and medication (e.g., chenodeoxycholic acids or ursodeoxycholic acid).

Methods disclosed herein include inducing rapid weight loss in a patient in need thereof, comprising administering to said patient a pharmaceutically effective amount of a MetAP2 inhibitor, wherein said patient has reduced incidence of hepatobiliary dysfunction, e.g., gallstones, as compared to a subject with rapid weight loss after treatment by a reduced energy diet or bariatric surgery. The disclosure is also related to a method of reducing the risk of gallstone formation in a patient being treated for obesity and undergoing rapid weight loss, comprising administering a pharmaceutically effective amount of a MetAP2 inhibitor. The disclosure is further related to a method of reducing hepatobiliary dysfunction, such as liver tumors or gallstones, as indicated by reduced alkaline phosphatase levels in a patient, whether or not the patient has undergone rapid weight loss, comprising administering a pharmaceutically effective amount of a MetAP2 inhibitor.

### MetAP2 Inhibitors

MetAP2 inhibitors refer to a class of molecules that inhibit or modulate the activity of MetAP2, e.g., the ability of MetAP2 to cleave the N-terminal methionine residue of newly synthesized proteins to produce the active form of the protein, or the ability of MetAP2 to regulate protein synthesis by protecting the subunit of eukaryotic initiation factor-2 (eIF2) and/or ERK1/2 from phosphorylation.

Exemplary MetAP2 inhibitors may include irreversible inhibitors that covalently bind to MetAP2. For example, such irreversible inhibitors include fumagillin, fumagillol, and fumagillin ketone.

Derivatives and analogs of fumagillin, and pharmaceutically acceptable salts thereof are contemplated herein as irreversible MetAP2 inhibitors, such as O-(4-dimethylaminoethoxycinnamoyl)fumagillol (also referred to herein as Compound A), O-(3,4,5-trimethoxycinnamoyl)fumagillol, O-(4-chlorocinnamoyl)fumagillol; O-(4-aminocinnamoyl)fumagillol; O-(4-dimethylaminoethoxycinnamoyl)fumagillol; O-(4-methoxycinnamoyl)fumagillol; O-(4-dimethylaminocinnamoyl)fumagillol; O-(4-hydroxycinnamoyl)fumagillol; O-(3,4-dimethoxycinnamoyl)fumagillol; O-(3,4-methylenedioxycinnamoyl)fumagillol; O-(3,4,5-trimethoxycinnamoyl)fumagillol; O-(4-nitrocinnamoyl)fumagillol; O-(3,4-dimethoxy-6-aminocinnamoyl)fumagillol; O-(4-acetoxy-3,5-dimethoxycinnamoyl)fumagillol; O-(4-ethylaminocinnamoyl)fumagillol; O-(4-ethylaminoethoxycinnamoyl)fumagillol; O-(3-dimethylaminomethyl-4-methoxycinnamoyl)fumagillol; O-(4-trifluoromethylcinnamoyl)fumagillol; O-(3,4-dimethoxy-6-nitrocinnamoyl)fumagillol; O-(4-acetoxycinnamoyl)fumagillol; O-(4-cyanocinnamoyl)fumagillol; 4-(4-methoxycinnamoyl)oxy-2-(1,2-epoxy-1,5-dimethyl-4-hexenyl)-3-methoxy-1-chloromethyl-1-cyclohexanol; O-(3,4,5-trimethoxycinnamoyl)fumagillol; O-(4-dimethylaminocinnamoyl)fumagillol; O-(3,4,5-trimethoxycinnamoyl)oxy-2-(1,2-epoxy-1,5-dimethyl-4-hexenyl)-3-m- ethoxy-1-chloromethyl-1-cyclohexanol; O-(4-dimethylaminocinnamoyl)oxy-2-(1,2-epoxy-1,5-dimethyl-4-hexenyl)-3-methoxy-1-chloromethyl-1-cyclohexanol; O-(3,5-dimethoxy-4-hydroxycinnamoyl)fumagillol or O-(chloracetyl-carbamoyl) fumagillol(TNP-470), and/or pharmaceutically acceptable salts thereof (e.g., O-(4-dimethylaminoethoxycinnamoyl)fumagillol oxalate).

Fumagillin, and some derivatives thereof, have a carboxylic acid moiety and can be administered in the form of the free acid. Alternatively, contemplated herein are pharmaceutically acceptable salts of fumagillin, fumagillol, and derivatives thereof.

Pharmaceutically acceptable salts illustratively include those that can be made using the following bases: ammonia, L-arginine, benethamine, benzathene, betaine, bismuth, calcium hydroxide, choline, deanol, diethanolamine, diethylamine, 2-(diethylamino)ethanol, ethylenediamine, N-methylglucarnine, hydrabamine, 1 H-imidazole, lysine, magnesium hydroxide, 4-(2-hydroxyethyl)morpholine, piperazine, potassium hydroxide, 1-(2-hydroxyethyl)pyrrolidine, sodium hydroxide, triethanolamine, zinc hydroxide, diclyclohexlamine, or any other electron pair donor (as described in Handbook of Pharmaceutical Salts, Stan & Wermuth, VHCA and Wiley, Uchsenfurt-Hohestadt Germany, 2002). Contemplated pharmaceutically acceptable salts may include hydrochloric acid, bromic acid, sulfuric acid, phosphoric acid, nitric acid, formic acid, acetic acid, trifluoroacetic acid, oxalic acid, fumaric acid, tartaric acid, maleic acid, methanesulfonic acid, benzenesulfonic acid or para-toluenesulfonic acid.

Esters of the present invention may be prepared by reacting fumagillin or fumagillol with the appropriate acid under standard esterification conditions described in the literature (Houben-Weyl 4th Ed. 1952, Methods of Organic Synthesis). Suitable fumagillin esters include ethyl methanoate, ethyl ethanoate, ethyl propanoate, propyl methanoate, propyl ethanoate, and methyl butanoate.

In another embodiment, contemplated irreversible inhibitors of MetAP2 may include a siRNA, shRNA, an antibody or an antisense compound of MetAP2.

Further examples of reversible and irreversible MetAP2 inhibitors are provided in the following references, each of which is hereby incorporated by reference: Olson et al. (U.S. Pat. No. 7,084,108 and WO 2002/042295), Olson et al. (U.S. Pat. No. 6,548,477; U.S. Pat. No. 7,037,890; U.S. Pat. No. 7,084,108; U.S. Pat. No. 7,268,111; and WO 2002/042295), Olson et al. (WO 2005/066197), Hong et al. (U.S. Pat. No. 6,040,337)., Hong et al. (U.S. Pat. No. 6,063,812 and WO 1999/059986), Lee et al. (WO 2006/080591), Kishimoto et al. (U.S. Pat. No. 5,166,172; U.S. Pat. No. 5,698,586; U.S. Pat. Nos. 5,164,410; and 5,180,738), Kishimoto et al. (U.S. Pat. No. 5,180,735), Kishimoto et al. (U.S. Pat. No. 5,288,722), Kishimoto et al. (U.S. Pat. No. 5,204,345), Kishimoto et al. (U.S. Pat. No. 5,422,363), Liu et al. (U.S. Pat. No. 6,207,704; U.S. Pat. No. 6,566,541; and WO 1998/056372), Craig et al. (WO 1999/057097), Craig et al. (U.S. Pat. No. 6,242,494), BaMaung et al. (U.S. Pat. No. 7,030,262), Comess et al. (WO 2004/033419), Comess et al. (US 2004/0157836), Comess et al. (US 2004/0167128), Henkin et al. (WO 2002/083065), Craig et al. (U.S. Pat. No. 6,887,863), Craig et al. (US 2002/0002152), Sheppard et al. (2004, Bioorganic & Medicinal Chemistry Letters 14:865-868), Wang et al. (2003, Cancer Research 63:7861-7869), Wang et al. (2007, Bioorganic & Medicinal Chemistry Letters 17:2817-2822), Kawai et al. (2006, Bioorganic & Medicinal Chemistry Letters 16:3574-3577), Henkin et al. (WO 2002/026782), Nan et al. (US 2005/0113420), Luo et al. (2003, J. Med. Chem., 46:2632-2640), Vedantham et al. (2008, J. Comb. Chem., 10:195-203), Wang et al. (2008, J. Med. Chem., XXXX, Vol. xxx, No. xx), Ma et al. (2007, BMC Structural Biology, 7:84) and Huang et al. (2007, J. Med. Chem., 50:5735-5742), Evdokimov et al. (2007, PROTEINS: Structure, Function, and Bioinformatics, 66:538-546), Garrabrant et al. (2004, Angiogenesis 7:91-96), Kim et al. (2004, Cancer Research, 64:2984-2987), Towbin et al. (2003, The Journal of Biological Chemistry, 278(52):52964-52971), Marino Jr. (U.S. Pat. No. 7,304,082), Kallender et al. (U.S. patent application number 2004/0192914), and Kallender et al. (U.S. patent application numbers 2003/0220371 and 2005/0004116). Other MetAP2 inhibitors contemplated herein are disclosed in U.S. Ser. Nos. 61/310,776; 61/293,318; 61/366,650 and PCT/US10/52050 (all of the above is hereby incorporated by reference in their entirety).

For example, contemplated MetAP2 inhibitors may include:

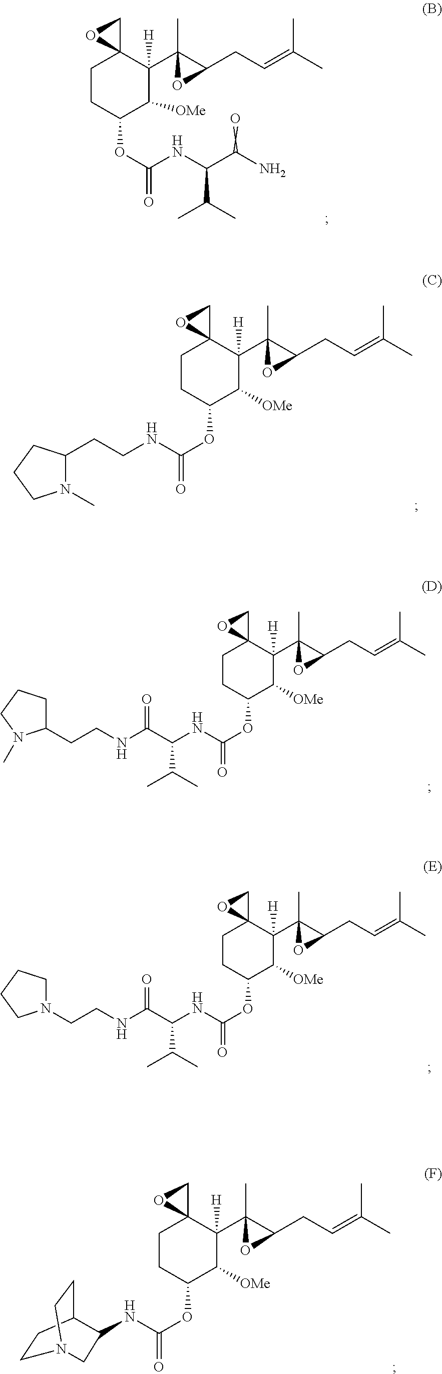

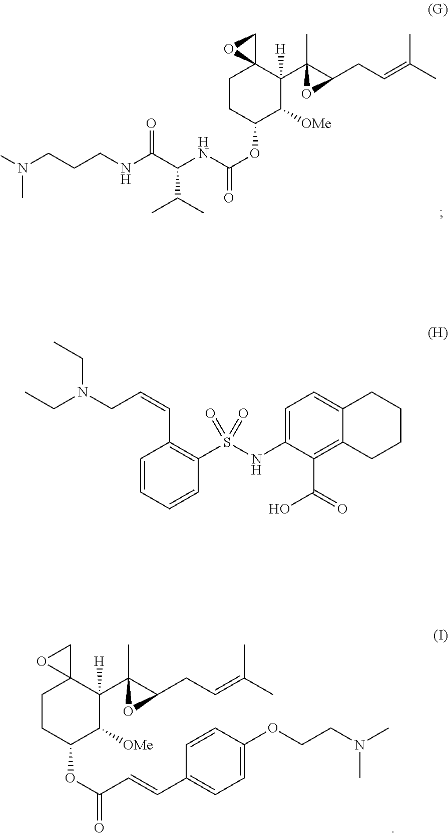

### Methods

A method of inducing rapid weight loss in a patient in need thereof, while reducing the risk of developing hepatobiliary dysfunction is provided herein that comprising administering a pharmaceutically effective amount of a MetAP2 inhibitor such as those provided herein. Hepatobiliary dysfunction may lead to any number of biliary tract disorders, including cholecystitis (inflammation of the gall bladder), biliary sludging, pancreatitis, liver tumors and cholelithiasis (gallstones). In an embodiment, a method of reducing the risk of gallstone formation in a patient being treated for obesity and undergoing rapid weight loss comprises administering a pharmaceutically effective amount of a MetAP2 inhibitor. Contemplated pharmaceutically effective amounts may not, in some embodiments, substantially modulate or suppress angiogenesis.

Patients contemplated for treatment of disclosed disorders or diseases include patients of normal weight, or an obese patient and/or a patient suffering from diabetes. In some embodiments, patients contemplated for treatment include those who are undergoing rapid weight loss (e.g., via a reduced energy diet or as a result of bariatric surgery). In other embodiments, patients contemplated for treatment include those who are suffering from hepatobiliary dysfunction with or without concurrent obesity, and who would benefit from treatment with a MetAP2 inhibitor.

In some embodiments, co-administration of a MetAP2 inhibitor and another active agent (e.g., a non-steroidal anti-inflammatory agent or ursodeoxycholic acid) occur at the same time. In other embodiments, administration of a MetAP2 inhibitor occurs immediately prior to or immediately administration of another active agent. In yet another embodiment, a period of time may elapse between administration of a MetAP2 inhibitor and another agent.

### Administration and Formulation

Contemplated herein are formulations suitable for parenteral or non-parenteral administration of MetAP2 inhibitors. In certain embodiments, a subject may have a lower systemic exposure (e.g., at least about 2, 3, 5, 10, 20, or at least about 30% less systemic exposure) to the non-parenterally (e.g., orally) administered of a MetAP2 inhibitor as compared to a subject parenterally (e.g., subcutaneously) administered the same dose of the MetAP2 inhibitor.

Contemplated non-parenteral administration includes oral, buccal, transdermal (e.g., by a dermal patch), topical, inhalation, sublingual, ocular, pulmonary, nasal, or rectal administration.

Contemplated parenteral administration includes intravenous and subcutaneous administration, as well as administration at a site of a minimally-invasive procedure or a surgery.

In another embodiment, provided herein are effective dosages, e.g., a daily dosage of a MetAP2 inhibitor, that may not substantially modulate or suppress angiogenesis. For example, provided here are methods that include administering doses of MetAP2 inhibitors that are effective for e.g., reducing lipids or cholesterol, but are significantly smaller doses than that necessary to modulate and/or suppress angiogenesis (which may typically require about 12.5 mg/kg to about 50 mg/kg or more). For example, contemplated dosage of a MetAP2 inhibitor in the methods described herein may include administering about 25 mg/day, about 10 mg/day, about 5 mg/day, about 3 mg/day, about 2 mg/day, about 1 mg/day, about 0.75 mg/day, about 0.5 mg/day, about 0.1 mg/day, about 0.05 mg/day, or about 0.01 mg/day.

For example, an effective amount of the drug for reducing cholesterol or lipids in a patient in need thereof may be about 0.0001 mg/kg to about 25 mg/kg of body weight per day. For example, a contemplated dosage may from about 0.001 to 10 mg/kg of body weight per day, about 0.001 mg/kg to 1 mg/kg of body weight per day, about 0.001 mg/kg to 0.1 mg/kg of body weight per day or about 0.005 to about 0.04 mg/kg or about 0.005 to about 0.049 mg/kg of body weight a day. In an embodiment a MetAP2 inhibitor such as disclosed herein (e.g., O-(4-dimethlyaminoethoxycinnamoyl)fumagillol), may be administered about 0.005 to about 1 mg/kg, or to about 5 mg/kg, or about 0.005 to about 0.1 mg/kg of a subject.

For example, provided herein is a method for treating or reducing the risk of a cardiovascular disease in a subject in need thereof, comprising administering, parenterally (e.g., intravenously) or non-parenterally, about 0.005 to about 1 mg/kg, or about 0.005 to about 1.0 mg/kg or to 0.005 to about 0.05 mg/kg of a MetAP2 inhibitor, selected from O-(4-dimethylaminoethoxycinnamoyl)fumagillol and pharmaceutically acceptable salts thereof (for example, an oxalate salt), to said subject.

Contemplated methods may include administration of a composition comprising a MetAP2 inhibitor, for example, hourly, twice hourly, every three to four hours, daily, twice daily, 1, 2, 3 or 4 times a week, every three to four days, every week, or once every two weeks depending on half-life and clearance rate of the particular composition or inhibitor.

Treatment can be continued for as long or as short a period as desired. The compositions may be administered on a regimen of, for example, one to four or more times per day. A suitable treatment period may be, for example, at least about one week, at least about two weeks, at least about one month, at least about six months, at least about 1 year, or indefinitely. A treatment regimen may include a corrective phase, during which a MetAP2 inhibitor dose sufficient to provide e.g., reduction of symptoms is administered, followed by a maintenance phase, during which a lower MetAP2 inhibitor dose sufficient to reduce or prevent increase in occurrence of symptoms of the treated disease is administered.

For pulmonary (e.g., intrabronchial) administration, MetAP2 inhibitors may be formulated with conventional excipients to prepare an inhalable composition in the form of a fine powder or atomizable liquid. For ocular administration, MetAP2 inhibitors may be formulated with conventional excipients, for example, in the form of eye drops or an ocular implant. Among excipients useful in eye drops are viscosifying or gelling agents, to minimize loss by lacrimation through improved retention in the eye.

Liquid dosage forms for oral or other administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the ocular, oral, or other systemically-delivered compositions can also include adjuvants such as wetting agents, and emulsifying and suspending agents.

Dosage forms for topical or transdermal administration of an inventive pharmaceutical composition may include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, cutaneous routes of administration are achieved with aqueous drops, a mist, an emulsion, or a cream.

Transdermal patches may have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

When administered in lower doses, injectable preparations for intravenous or subcutaneous administration are also contemplated herein, for example, sterile injectable aqueous or oleaginous suspensions may be formulated according to the known art using suitable dispersing or wetting agents and suspending agents.

Compositions for rectal administration may be suppositories which can be prepared by mixing a MetAP2 inhibitor with suitable non-irritating excipients or carriers such as cocoa butter, polyethylene glycol or a suppository wax which are solid at ambient temperature but liquid at body temperature and therefore melt in the rectum and release the active agent(s). Alternatively, contemplated formulations can be administered by release from a lumen of an endoscope after the endoscope has been inserted into a rectum of a subject.

Oral dosage forms, such as capsules, tablets, pills, powders, and granules, may be prepared using any suitable process known to the art. For example, a MetAP2 inhibitor may be mixed with enteric materials and compressed into tablets.

Alternatively, formulations of the invention are incorporated into chewable tablets, crushable tablets, tablets that dissolve rapidly within the mouth, or mouth wash.

### EXAMPLES

The following examples are not intended in any way to limit the scope of this invention but is provided to illustrate aspects of the disclosed methods. Many other embodiments of this invention will be apparent to one skilled in the art.

### Example 1

### Administration of MetAP2 Inhibitor Reduces Alkaline Phosphatase

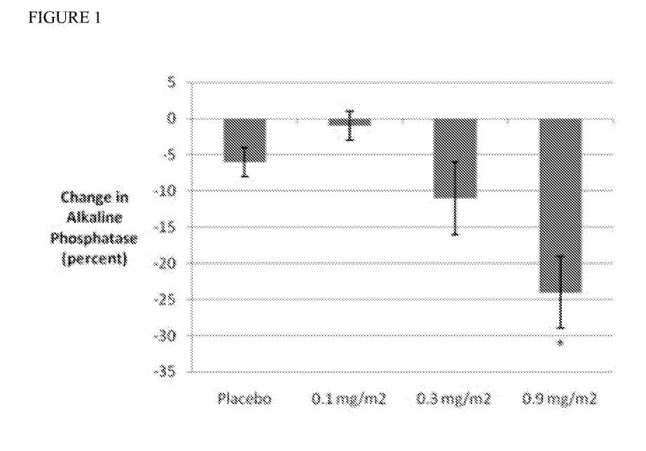

Patients treated with either a placebo, 0.1 mg/m² (approximately 0.2 mg per dose) of the MetAP2 inhibitor O-(4-dimethylaminoethoxycinnamoyl)fumagillol, 0.3 mg/m² (approximately 0.6 mg per dose), or 0.9 mg/m² of calculated body surface area (approximately 1.9 mg per dose) administered biweekly for 26 days. A significant reduction in alkaline phosphatase was observed with increasing MetAP2 inhibitor dosage (FIG. 1). This reduction is indicative of a decrease in hepatic injury or hepatobiliary dysfunction, which is consistent with reduced biliary sludging and/or gallstone formation.

### Incorporation by Reference

References and citations to other documents, such as patents, patent applications, patent publications, journals, books, papers, web contents, have been made throughout this disclosure. All such documents are hereby incorporated herein by reference in their entirety for all purposes, including PCT/US09/66816.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. (canceled)

2. (canceled)

3. A method of reducing the risk of gallstone formation in a patient being treated for obesity and undergoing rapid weight loss, comprising administering a pharmaceutically effective amount of a MetAP2 inhibitor.

4. (canceled)

5. A method of reducing hepatobiliary dysfunction, as indicated by reduced alkaline phosphatase levels in a patient undergoing rapid weight loss, comprising administering a pharmaceutically effective amount of a MetAP2 inhibitor.

6. The method of claim 3, wherein the rapid weight loss is at least about 15 kg after 19 weeks of treatment with the MetAP2 inhibitor.

7. The method of claim 3, wherein the rapid weight loss is at least about 20 kg after 25 weeks of treatment with the MetAP2 inhibitor.

8. (canceled)

9. (canceled)

10. A method of treating gallstones in a patient in need thereof, comprising administering a pharmaceutically effective amount of a MetAP2 inhibitor.

11. The method of claim 10, wherein the patient is being treated for obesity and undergoing rapid weight loss.

12. The method of claim 3, wherein the patient is human.

13. The method of claim 3, wherein the patient is female.

14. The method of claim 12, wherein the patient has a Body Mass Index measurement of at least about 25 kg/m², at least about 30 kg/m², or at least about 40 kg/m².

15. The method of claim 1, wherein the patient is a cat or a dog.

16. The method of claim 1, wherein said pharmaceutically effective amount does not substantially modulate or suppress angiogenesis.

17. The method of claim 1, wherein said MetAP2 inhibitor is a substantially irreversible inhibitor.

18. The method of claim 1, wherein said MetAP2 inhibitor is selected from the group consisting of a fumagillin, fumagillol or fumagillin ketone, or a derivative thereof, siRNA, shRNA, an antibody, or an antisense compound.

19. The method of claim 1, wherein said MetAP2 inhibitor is selected from O-(4-dimethylaminoethoxycinnamoyl)fumagillol and pharmaceutically acceptable salts thereof.

20. The method of claim 1, wherein said MetAP2 inhibitor is a substantially reversible inhibitor.

21. The method of claim 1, further comprising administering to said patient a pharmaceutically acceptable amount of a non-steroidal anti-inflammatory agent or ursodeoxycholic acid.
